(19) United States
(12) Patent Application Publication
(10) Pub. No.: US 2021/0210212 A1
(43) Pub. Date: Jul. 8, 2021

(54) Health Change Prediction Based on Internet of Things Data

(71) Applicant: International Business Machines Corporation, Armonk, NY (US)

(72) Inventors: Craig M. Trim, Ventura, CA (US); Zachary A. Silverstein, Jacksonville, FL (US); Sarbajit K. Rakshit, Kolkata (IN); Maruthi Prasad Koiloth, Little Elm, TX (US)

(21) Appl. No.: 16/733,925

(22) Filed: Jan. 3, 2020

(51) Int. Cl. G16H 50/70; H04L 29/08

(52) U.S. Cl. CPC G16H 50/70; G16Y 10/60; H04L 67/306; H04L 67/12

(57) ABSTRACT

Detecting illness onset of a user based on IoT data is provided. Onset of an illness affecting the user is detected based on analyzing data regarding the user collected from a plurality of IoT devices corresponding to the user. Crowdsourced data is collected from a defined area surrounding a geolocation of the user. The crowdsourced data is related to the illness and includes a median illness duration and severity for the illness. An illness history of the user is collected from a profile corresponding to the user. Criticality of upcoming events is identified in an electronic calendar corresponding to the user. A recommended course of action for the user regarding the illness is calculated based on the crowdsourced data from the defined area surrounding the geolocation of the user, the illness history of the user, and the criticality of upcoming events corresponding to the user.

### BACKGROUND

### 1. Field

The disclosure relates generally to Internet of Things and more specifically to predicting health change of a user based on Internet of Things data.

### 2. Description of the Related Art

The Internet of Things (IoT) is a system of interrelated data processing devices provided with unique identifiers and an ability to transfer data over a network without requiring human-to-device interaction. IoT technology is most synonymous with devices pertaining to a “smart home”, which may include, for example, smart appliances, smart cabinets, smart lighting fixtures, smart thermostats, smart home monitoring systems, smart cameras, smart microphones, and the like. IoT devices can be used to enable remote health monitoring and emergency notifications. These IoT health monitoring devices can range from blood pressure monitors, heart rate monitors, pacemaker monitors, activity monitors, speech monitors, and the like.

Understanding change in health and illness over time is central to evaluating interventions for individuals. Understanding the course of change in health over time allows anticipation of those at risk for illness, enhances understanding of factors that influence change in health over time, and permits examination of the effects of treatment. Thus, knowledge about the course of change in health status over time creates the possibility for controlling self-care to impact health over time.

### SUMMARY

According to one illustrative embodiment, a computer-implemented method for detecting illness onset of a user based on IoT data is provided. Onset of an illness affecting the user is detected based on analyzing data regarding the user collected from a plurality of IoT devices corresponding to the user. Crowdsourced data is collected from a defined area surrounding a geolocation of the user. The crowdsourced data is related to the illness of the user and includes a median illness duration and severity for the illness. An illness history of the user is collected from a profile corresponding to the user. Criticality of upcoming events is identified in an electronic calendar corresponding to the user. A recommended course of action for the user regarding the illness is calculated based on the crowdsourced data from the defined area surrounding the geolocation of the user, the illness history of the user, and the criticality of upcoming events corresponding to the user. According to other illustrative embodiments, a computer system and computer program product for detecting illness onset of a user based on IoT data are provided.

### BRIEF DESCRIPTION OF THE DRAWINGS

FIG. 1 is a pictorial representation of a network of data processing systems in which illustrative embodiments may be implemented;

FIG. 2 is a diagram of a data processing system in which illustrative embodiments may be implemented;

FIG. 3 is a diagram illustrating an example of an illness monitoring system in accordance with an illustrative embodiment;

FIG. 4 is a flowchart illustrating a process for analyzing symptom-correlated data corresponding to a user in accordance with an illustrative embodiment;

FIGS. 5A-5B are a flowchart illustrating a process for predicting a course of action for a user regarding an illness in accordance with an illustrative embodiment; and

FIG. 6 is a flowchart illustrating a process for detecting illness onset of a user based on IoT data in accordance with an illustrative embodiment.

### DETAILED DESCRIPTION

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

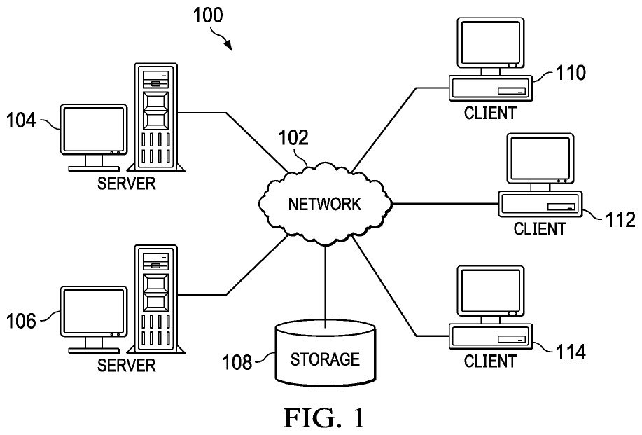

With reference now to the figures, and in particular, with reference to FIGS. 1-3, diagrams of data processing environments are provided in which illustrative embodiments may be implemented. It should be appreciated that FIGS. 1-3 are only meant as examples and are not intended to assert or imply any limitation with regard to the environments in which different embodiments may be implemented. Many modifications to the depicted environments may be made.

FIG. 1 depicts a pictorial representation of a network of data processing systems in which illustrative embodiments may be implemented. Network data processing system 100 is a network of computers, data processing systems, and other devices, such as, for example, Internet of Things (IoT) devices, in which the illustrative embodiments may be implemented. Network data processing system 100 contains network 102, which is the medium used to provide communications links between the computers, data processing systems, and other devices connected together within network data processing system 100. Network 102 may include connections, such as, for example, wire communication links, wireless communication links, fiber optic cables, and the like.

In the depicted example, server 104 and server 106 connect to network 102, along with storage 108. Server 104 and server 106 may be, for example, server computers with high-speed connections to network 102. In addition, server 104 and server 106 provide illness monitoring services to registered users. Also, it should be noted that server 104 and server 106 may each represent multiple computing nodes in one or more cloud environments providing illness monitoring services. Alternatively, server 104 and server 106 may each represent a cluster of servers in one or more data centers.

Client 110, client 112, and client 114 also connect to network 102. Clients 110, 112, and 114 are clients of server 104 and server 106. In this example, clients 110, 112, and 114 are shown as desktop or personal computers with wire communication links to network 102. However, it should be noted that clients 110, 112, and 114 are examples only and may represent other types of data processing systems, such as, for example, laptop computers, handheld computers, smart phones, smart watches, smart televisions, and the like, with wire or wireless communication links to network 102. In this example, users of clients 110, 112, and 114 are registered users of the illness monitoring services provided by server 104 and server 106. The registered users may utilize clients 110, 112, and 114 to receive illness duration and severity notifications, course of treatment recommendations for an illness, and treatment escalation alerts from server 104 and server 106.

Storage 108 is a network storage device capable of storing any type of data in a structured format or an unstructured format. In addition, storage 108 may represent a plurality of network storage devices. Further, storage 108 may store identifiers for a plurality of different registered users, historical illness profiles corresponding to the registered users, identifiers and network addresses of a set of IoT devices corresponding to each respective registered user, crowdsourced illness duration and severity data for a plurality of different defined areas, and the like. Furthermore, storage 108 may store other types of data, such as authentication or credential data that may include user names, passwords, and biometric templates associated with system administrators and registered users, for example.

In addition, it should be noted that network data processing system 100 may include any number of additional servers, clients, storage devices, and other devices not shown. Program code located in network data processing system 100 may be stored on a computer readable storage medium and downloaded to a computer or other data processing device for use. For example, program code may be stored on a computer readable storage medium on server 104 and downloaded to client 110 over network 102 for use on client 110.

In the depicted example, network data processing system 100 may be implemented as a number of different types of communication networks, such as, for example, an internet, an intranet, a personal area network (PAN), a local area network (LAN), a wide area network (WAN), a telecommunications network, or any combination thereof. FIG. 1 is intended as an example only, and not as an architectural limitation for the different illustrative embodiments.

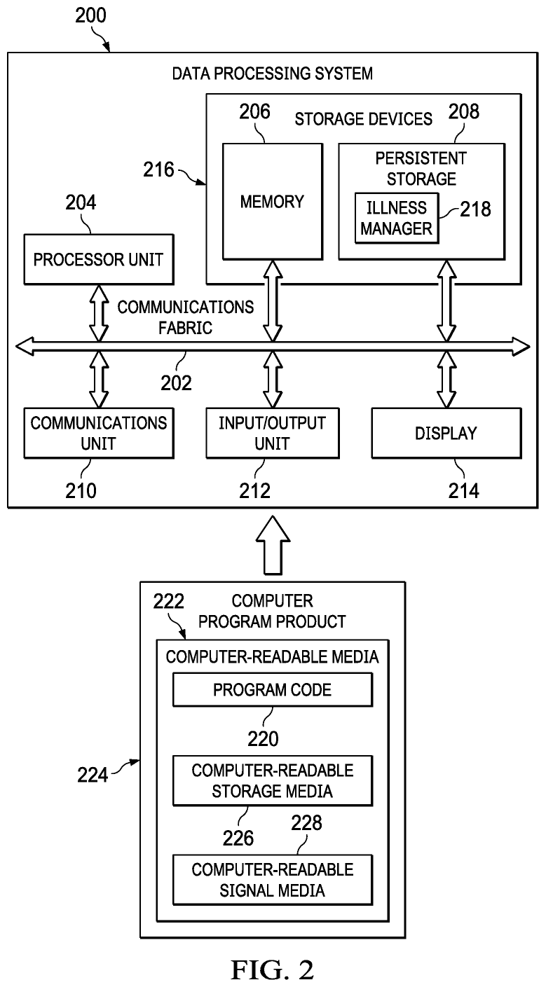

With reference now to FIG. 2, a diagram of a data processing system is depicted in accordance with an illustrative embodiment. Data processing system 200 is an example of a computer, such as server 104 in FIG. 1, in which computer readable program code or instructions implementing processes of illustrative embodiments may be located. In this example, data processing system 200 includes communications fabric 202, which provides communications between processor unit 204, memory 206, persistent storage 208, communications unit 210, input/output (I/O) unit 212, and display 214.

Processor unit 204 serves to execute instructions for software applications and programs that may be loaded into memory 206. Processor unit 204 may be a set of one or more hardware processor devices or may be a multi-core processor, depending on the particular implementation.

Memory 206 and persistent storage 208 are examples of storage devices 216. A computer readable storage device is any piece of hardware that is capable of storing information, such as, for example, without limitation, data, computer readable program code in functional form, and/or other suitable information either on a transient basis or a persistent basis. Further, a computer readable storage device excludes a propagation medium. Memory 206, in these examples, may be, for example, a random-access memory (RAM), or any other suitable volatile or non-volatile storage device, such as a flash memory. Persistent storage 208 may take various forms, depending on the particular implementation. For example, persistent storage 208 may contain one or more devices. For example, persistent storage 208 may be a disk drive, a solid-state drive, a rewritable optical disk, a rewritable magnetic tape, or some combination of the above. The media used by persistent storage 208 may be removable. For example, a removable hard drive may be used for persistent storage 208.

In this example, persistent storage 208 stores illness manager 218. However, it should be noted that even though illness manager 218 is illustrated as residing in persistent storage 208, in an alternative illustrative embodiment illness manager 218 may be a separate component of data processing system 200. For example, illness manager 218 may be a hardware component coupled to communication fabric 202 or a combination of hardware and software components. In another alternative illustrative embodiment, a first set of components of illness manager 218 may be located in data processing system 200 and a second set of components of illness manager 218 may be located in a second data processing system, such as, for example, server 106 or client 110 in FIG. 1.

Illness manager 218 controls the process of analyzing symptom-correlated data corresponding to a registered user, which is collected from a plurality of IoT devices corresponding to the user, to detect illness of the user. Further, illness manager 218 utilizes crowdsourced illness duration and severity data collected from a defined area surrounding a geolocation of the user, illness duration and severity history of the user, and scheduled upcoming critical events and tasks for the user collected from an electronic calendar to determine whether the user is suffering from similar or unexpected symptoms, to predict how the illness will impact productivity of the user taking into account effect of treatment on the upcoming critical events and tasks, and to calculate a recommended course of action for the user with respect to the illness to ameliorate the impact of the illness on the user.

As a result, data processing system 200 operates as a special purpose computer in which illness manager 218 in data processing system 200 enables prediction of illness duration, severity, and productivity impact on a user. In particular, illness manager 218 transforms data processing system 200 into a special purpose computer system as compared to currently available general computer systems that do not have illness manager 218.

Communications unit 210, in this example, provides for communication with other computers, data processing systems, and devices via a network, such as network 102 in FIG. 1. Communications unit 210 may provide communications through the use of both physical and wireless communications links. The physical communications link may utilize, for example, a wire, cable, universal serial bus, or any other physical technology to establish a physical communications link for data processing system 200. The wireless communications link may utilize, for example, shortwave, high frequency, ultrahigh frequency, microwave, wireless fidelity (Wi-Fi), Bluetooth® technology, global system for mobile communications (GSM), code division multiple access (CDMA), second-generation (2G), third-generation (3G), fourth-generation (4G), 4G Long Term Evolution (LTE), LTE Advanced, fifth-generation (5G), or any other wireless communication technology or standard to establish a wireless communications link for data processing system 200.

Input/output unit 212 allows for the input and output of data with other devices that may be connected to data processing system 200. For example, input/output unit 212 may provide a connection for user input through a keypad, a keyboard, a mouse, a microphone, and/or some other suitable input device. Display 214 provides a mechanism to display information to a user and may include touch screen capabilities to allow the user to make on-screen selections through user interfaces or input data, for example.

Instructions for the operating system, applications, and/or programs may be located in storage devices 216, which are in communication with processor unit 204 through communications fabric 202. In this illustrative example, the instructions are in a functional form on persistent storage 208. These instructions may be loaded into memory 206 for running by processor unit 204. The processes of the different embodiments may be performed by processor unit 204 using computer-implemented instructions, which may be located in a memory, such as memory 206. These program instructions are referred to as program code, computer usable program code, or computer readable program code that may be read and run by a processor in processor unit 204. The program instructions, in the different embodiments, may be embodied on different physical computer readable storage devices, such as memory 206 or persistent storage 208.

Program code 220 is located in a functional form on computer readable media 222 that is selectively removable and may be loaded onto or transferred to data processing system 200 for running by processor unit 204. Program code 220 and computer readable media 222 form computer program product 224. In one example, computer readable media 222 may be computer readable storage media 226 or computer readable signal media 228. Computer readable storage media 226 may include, for example, an optical or magnetic disc that is inserted or placed into a drive or other device that is part of persistent storage 208 for transfer onto a storage device, such as a hard drive, that is part of persistent storage 208. Computer readable storage media 226 also may take the form of a persistent storage, such as a hard drive, a thumb drive, or a flash memory that is connected to data processing system 200. In some instances, computer readable storage media 226 may not be removable from data processing system 200.

Alternatively, program code 220 may be transferred to data processing system 200 using computer readable signal media 228. Computer readable signal media 228 may be, for example, a propagated data signal containing program code 220. For example, computer readable signal media 228 may be an electro-magnetic signal, an optical signal, and/or any other suitable type of signal. These signals may be transmitted over communication links, such as wireless communication links, an optical fiber cable, a coaxial cable, a wire, and/or any other suitable type of communications link. In other words, the communications link and/or the connection may be physical or wireless in the illustrative examples. The computer readable media also may take the form of non-tangible media, such as communication links or wireless transmissions containing the program code.

In some illustrative embodiments, program code 220 may be downloaded over a network to persistent storage 208 from another device or data processing system through computer readable signal media 228 for use within data processing system 200. For instance, program code stored in a computer readable storage media in a data processing system may be downloaded over a network from the data processing system to data processing system 200. The data processing system providing program code 220 may be a server computer, a client computer, or some other device capable of storing and transmitting program code 220.

The different components illustrated for data processing system 200 are not meant to provide architectural limitations to the manner in which different embodiments may be implemented. The different illustrative embodiments may be implemented in a data processing system including components in addition to, or in place of, those illustrated for data processing system 200. Other components shown in FIG. 2 can be varied from the illustrative examples shown. The different embodiments may be implemented using any hardware device or system capable of executing program code. As one example, data processing system 200 may include organic components integrated with inorganic components and/or may be comprised entirely of organic components excluding a human being. For example, a storage device may be comprised of an organic semiconductor.

As another example, a computer readable storage device in data processing system 200 is any hardware apparatus that may store data. Memory 206, persistent storage 208, and computer readable storage media 226 are examples of physical storage devices in a tangible form.

In another example, a bus system may be used to implement communications fabric 202 and may be comprised of one or more buses, such as a system bus or an input/output bus. Of course, the bus system may be implemented using any suitable type of architecture that provides for a transfer of data between different components or devices attached to the bus system. Additionally, a communications unit may include one or more devices used to transmit and receive data, such as a modem or a network adapter. Further, a memory may be, for example, memory 206 or a cache such as found in an interface and memory controller hub that may be present in communications fabric 202.

When people become ill, they ask basic questions, such as, for example, “How long is this illness going to last?”, “How bad is this going to get?”, “What else can I expect?”, and the like. Illustrative embodiments leverage advancements in IoT technology to answer these questions and provide a user with actionable information.

In addition, when people get sick from specific medical issues, they tend to be concerned with disease diagnosis, disease duration, and any related complications. Currently, approaches exist for determining the disease, itself, but determining disease duration and related complications is lacking in these current approaches. Illustrative embodiments do not make a medical diagnosis, but instead utilize IoT data corresponding to users of a defined area to predict duration and severity of minor and common illnesses, such as, for example, influenza, and provide recommended courses of treatment for these illnesses.

Illustrative embodiments utilize crowdsourced information from IoT data feeds clustered to each defined geographic area, such as, for example, a city, a county, a state, a region, a country, a continent, or the like, to derive estimated duration and severity of an illness. In a temporal constrained period, such as, for example, a week, a month, a quarter, a season, or the like, with a defined start and end time, illustrative embodiments receive symptom-correlated data indicative of illness from IoT devices. The symptom-correlated data may be, for example, coughing or sneezing audio signals detected by IoT microphones, products, such as medication, electrolyte drinks, cough drops, and the like, detected by smart storage, such as a smart refrigerator or smart cabinet, and permitted personal information, such as biometric data (e.g., heart rate, temperature, breaths per minute, perspiration, eye dilation, and the like), captured by IoT health monitoring devices, wearables, or implants.

Illustrative embodiments crowdsource all of this information by each defined geographic area so that each defined geographic area has a relative diagnosis of an illness and estimated median duration time and severity level for the illness. Illustrative embodiments estimate the impact of the illness on a user using historical learning and forward prediction. For example, illustrative embodiments capture historical learning on user illness attributes, such as how long did it take the user to recover from this illness previously, how did the illness impact the user's productivity, did treatment, such as increased rest, decrease the user's recovery time, and the like based on historical IoT data. Further, illustrative embodiments capture future prediction by accessing the user's electronic calendar to, for example, identify work-related entries, determine critical or urgent upcoming events or tasks, and the like. Based on the historical learning and the forward prediction, illustrative embodiments provide a recommendation as to whether or not the user should, for example, try to “tough it out” and remain active or take bed rest for a few days.

Furthermore, illustrative embodiments continue to capture symptom-correlated data from the IoT sensors for possible escalation. For example, illustrative embodiments continue capturing the user's symptoms and determine whether symptom severity exceeds a defined critical symptom severity threshold level. The defined critical symptom threshold may either be set manually by the user or set automatically by illustrative embodiments using machine learning. Moreover, illustrative embodiments monitor for detection of new symptoms in the IoT data. Additionally, illustrative embodiments may capture user symptoms through other channels, such as, for example, search engine results, social media posts, text messages, and the like, which correspond to the user. Illustrative embodiments determine whether the severity of current symptoms (e.g., is greater than the defined critical symptom severity threshold level) warrants escalation. For example, if the user coughs violently for 5 minutes, sleeps 15 hours a day, has diarrhea for more than 6 hours, vomits more than 7 times, or other similar symptoms, then illustrative embodiments send an escalation notification to the user or an emergency contact, such as a designated person or medical professional, for a proper response. If illustrative embodiments detect a new symptom, then illustrative embodiments predict whether the new symptom is out of the ordinary for this illness or is typical for this illness. If the new symptom is out of the ordinary, then illustrative embodiments send an extra notification and log the new symptom in the user's illness history profile.

As an example use case scenario, a user opts to join the illness monitoring service of illustrative embodiments. At some point, the user begins to cough above a defined threshold level and illustrative embodiments detect this as an onset of an illness. Illustrative embodiments detect the change in symptom-correlated data received from IoT devices corresponding to the user to determine the onset of the illness. Finally, illustrative embodiments detect that the user is feeling better when the user's symptoms fall below the defined threshold level. Illustrative embodiments, within a constrained time period, detect this information at a crowdsourced level between the user and many other users in the defined area surrounding the current geolocation of the user. Illustrative embodiments process this crowdsourced information to determine, for example, median or average symptom duration and severity for this illness and other details.

Also in this example use case scenario, a second user becomes ill in the same defined area surrounding the current location of the first user. Illustrative embodiments retrieve historical illness information for the second user from a profile corresponding to the second user. Based on the retrieved historical illness information, illustrative embodiments know the typical impact that illness has on the second user's productivity, sleep patterns, daily activities/behavior, and the like. As the second user crosses the defined threshold level, illustrative embodiments search an electronic calendar in one or more of the second user's IoT devices, such as smart phone or computer, for scheduled upcoming events and tasks. Illustrative embodiments predict the impact on the second user's productivity and wellbeing based on the historical illness information of the second user, crowdsourced information for the defined area, and upcoming events and tasks for the second user. Illustrative embodiments provide a recommendation to the second user regarding whether it would better for the second user to, for example, rest now and take a 1 day hit in productivity or continue to work at a 50% productivity level for next 4 days. If illustrative embodiments detect that the second user's symptom severity has crossed a second defined threshold level or that the second user has a new symptom that was not expected based on the historical illness information of the second user and the crowdsourced information for the defined area, then illustrative embodiments alert the second user to seek additional medical care as soon as possible, for example.

After a user opts in or registers with the illness monitoring service of illustrative embodiments, illustrative embodiments connect to the IoT devices corresponding to the user via a network. These IoT devices may include, for example, smart phones, smart watches, personal computers, laptop computers, handheld computers, smart televisions, smart appliances, smart storage devices, smart cameras, smart microphones, virtual agents, and the like. These IoT devices look for symptom-correlated data, such as changes in the user's daily activities/behavior, indicative of illness. Further, these IoT devices may utilize a natural language classifier or natural language understanding to identify symptom-correlated information, such as, for example, the user saying “I have a cold.” A smart microphone may pick up contextual audio of coughing severity and frequency after detecting the user saying “I'm sick.” A smart camera may detect the user opening a medicine bottle and taking a pill. A smart biometric device, such as a smart watch or activity monitor, may determine that the user is sleeping beyond the user's normal sleeping pattern. These IoT devices may anonymize this information and transmit this information, along with geolocation data, to illustrative embodiments for analysis.

Illustrative embodiments build a model that is time constrained for the illness within the defined area surrounding the geolocation of the user. Illustrative embodiments use this model to determine where the user is within the lifecycle of the illness, along with expected duration and severity of the illness. Illustrative embodiments derive the median illness duration and severity from a predictive model. Illustrative embodiment detect that the user is “officially” ill when the user goes above the defined illness symptom threshold level based on the user's own historical illness profile and median illness profile of other users in the defined area surrounding the geolocation of the user. The historical illness profile includes, for example, personal recovery time of the user for a normal or average illness, which is how long it takes the user to fall below the defined illness symptom threshold level.

Illustrative embodiments also determine the effect on user productivity by capturing how the user's work is impacted by the illness. Furthermore, illustrative embodiments determine the impact of treatment, such as medicine, bed rest, increased fluids, and the like, by the user on the lifecycle of the illness. Illustrative embodiments utilize, for example, natural language processing to detect upcoming critical events and tasks scheduled in the user's electronic calendar during the expected duration of the illness. Illustrative embodiments utilize all of the collected IoT data corresponding to the user and crowdsourced data for the defined area surrounding the geolocation of the user to generate a recommendation that will let the user know what the optimum course of action for the illness will be taking into account upcoming critical events and tasks.

If illustrative embodiments detect a new symptom or severity of a current symptom is increasing, then illustrative embodiments perform additional notification steps. If illustrative embodiments detect a new symptom and the new symptom is unexpected for the user, then illustrative embodiments add the new symptom to the historic illness record of the user and implement an alert protocol. If illustrative embodiments detect that the severity of a current symptom has increased above a defined critical illness threshold level, then illustrative embodiments notify the user that follow up action for medical attention is recommended. Moreover, illustrative embodiments may automatically notify medical professionals regarding the user's increased symptomatology above the defined critical illness threshold level.

Thus, illustrative embodiments analyze data regarding the user collected from a plurality of IoT devices to detect illness of the user and utilize collected crowdsourced data, illness history of the user, and scheduled critical events and tasks for the user to determine whether the user is suffering from similar or unexpected symptoms, to predict how the illness will impact the user taking into account effect of treatment on upcoming critical events and tasks, and to calculate a recommended course of action for the user with respect to the illness to ameliorate the impact of the illness on the user.

Thus, illustrative embodiments provide one or more technical solutions that overcome a technical problem with determining illness duration and severity for a user and impact on user productivity based on IoT data. As a result, these one or more technical solutions provide a technical effect and practical application in the field of IoT.

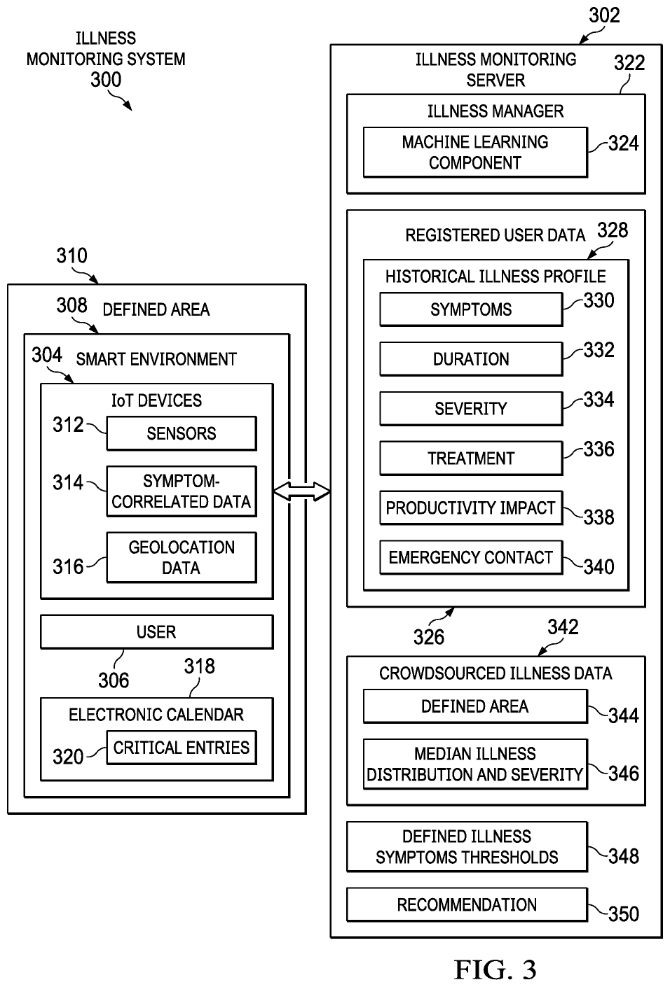

With reference now to FIG. 3, a diagram illustrating an example of an illness monitoring system is depicted in accordance with an illustrative embodiment. Illness monitoring system 300 may be implemented in a network of data processing systems, such as network data processing system 100 in FIG. 1. Illness monitoring system 300 is a system of hardware and software components for analyzing symptom-correlated data corresponding to a user collected from a plurality of IoT devices to detect illness of the user and predict duration, severity, and impact of the illness on the user.

In this example, illness monitoring system 300 includes illness monitoring server 302 and IoT devices 304. Illness monitoring server 302 may be, for example, server 104 in FIG. 1 or data processing system 200 in FIG. 2. IoT devices 304 represent a plurality of IoT devices corresponding to user 306. IoT devices 304 may include, for example, one or more computers, smart phones, smart watches, smart rings, smart bracelets, smart clothing, smart glasses, smart shoes, smart implants, smart beds, smart appliances, smart thermostats, smart storage, smart cameras, smart microphones, smart home monitoring systems, activity monitors, and the like. User 306 is a registered user of the illness monitoring services provided by illness monitoring server 302.

IoT devices 304 and user 306 are located in smart environment 308. Smart environment 308 may be, for example, a smart home, a smart office, a smart vehicle, or the like. In addition, smart environment 308 is located in defined area 310. Defined area 310 is an area or zone with a delineated boundary surrounding a geographic location of user 306. For example, defined area 310 may be neighborhood, a city block, a city, a county, a state, a country, or the like. Also, it should be noted that defined area 310 includes a plurality of different smart environments containing different sets of IoT devices corresponding to different registered users. Further, it should be noted that defined area 310 may represent a plurality of different defined areas within, for example, a city, state, region, country, continent, hemisphere, or the like.

IoT devices 304 include sensors 312. Sensors 312 may include, for example, heart rate sensors, breath rate sensors, body temperature sensors, body hydration sensors, activity sensors, accelerometers, motion sensors, pupil dilation sensors, perspiration sensors, image sensors, sound sensors, geolocation sensors, and the like. IoT devices 304 utilize sensors 312 to recognize and collect symptom-correlated data 314. Symptom-correlated data 314 relates to, or is indicative of, an illness, such as, for example, the flu, a migraine, bacterial infection, food poisoning, or the like. Symptom-correlated data 314 may be, for example, coughing or sneezing, groaning, crying, abdominal muscle cramping, increased body temperature, constricted pupils, eyes closed for prolonged periods during the day, perspiration at rest, increase heart and respiratory rate, prolonged sleep, reddening of the skin, increase intestinal sounds, user saying “I don't feel well”, and the like.

IoT devices 304 also provide geolocation data 316. One or more of IoT devices 304 may include, for example, a global positioning system (GPS) transceiver for providing geographic coordinates. Geolocation data 316 identifies the geographic location of user 306 within defined area 310.

In addition, one or more of IoT devices 304 store electronic calendar 318. Electronic calendar 318 corresponds to user 306. In this example, electronic calendar 318 includes critical entries 320. Critical entries 320 represent important, significant, or crucial events and tasks for user 306. User 306 may mark these specific events and tasks as critical when making corresponding entries in electronic calendar 318.

Illness monitoring server 302 includes illness manager 322, such as illness manager 218 in FIG. 2. In this example, illness manager 322 includes machine learning component 324. Machine learning component 324 may be, for example, an artificial intelligence software application capable of natural language processing, computer vision, data mining, exploratory data analysis, unsupervised learning, predictive analytics, and the like. Illness manager 322 utilizes machine learning component 324 to analyze symptom-correlated data 314 and critical entries 320 corresponding to user 306, historical illness profile 328, and crowdsourced illness data 342 to help formulate recommendation 350.

Registered user data 326 corresponds to user 306. Registered user data 326 may include, for example, a unique identifier for user 306 to uniquely identify user 306 anonymously. Registered user data 326 may also include identifiers for IoT devices 304. For example, user 306 may identify which particular IoT devices corresponding to user 306 that illness monitoring server 302 may connect to for collecting symptom-correlated data 314, geolocation data 316, and critical entries 320 corresponding to user 306.

Historical illness profile 328 corresponds to user 306 and includes a record of past illnesses of user 306. In this example, historical illness profile 328 includes symptoms 330, duration 332, severity 334, treatment 336, productivity impact 338, and emergency contact 340. However, it should be noted that historical illness profile 328 may include more or less information than shown.

Symptoms 330 identify the type of symptoms exhibited by user 306 during each particular past illness episode. Duration 332 identifies how long each particular past illness episode lasted in terms of hours, days, weeks, or the like. Severity 334 identifies a level of acuteness, seriousness, or significance each particular past illness episode. Treatment 336 identifies the type of treatment, such as, for example, medication, vitamin C, chicken soup, aspirin, antacids, fluids, and the like, used by user 306 during each particular past illness episode. Productivity impact 338 identifies the amount of influence or effect on the productivity or efficiency of user 306 on events and tasks during each particular past illness episode. Emergency contact 340 identifies one or more people, such as a spouse, relative, medical professional, and the like, that illness manager 302 should automatically notify when illness manager 322 detects a crisis or dangerous health situation related to user 306.

Crowdsourced illness data 342 represents symptom-correlated data indicative of illnesses collected and aggregated from a plurality of different sets of IoT devices corresponding to a plurality of different users within defined area 344. In this example, defined area 344 identifies defined area 310 in which user 306 is located. However, it should be noted that defined area 344 may identify any defined area utilizing the illness monitoring services of illness monitoring server 302.

Machine learning component 324 derives median illness duration and severity 346 from crowdsourced illness data 342 collected from defined area 344. Median illness duration and severity 346 identifies an average or typical duration and severity of one or more illnesses effecting registered users in defined area 344 within a time constrained period, such as a week, month, season, or the like.

Illness manager 322 utilizes defined illness symptom thresholds 348 to determine when user 306 is ill. For example, when symptom-correlated data 314 exceeds defined illness symptom thresholds 348, illness manager 322 determines that user 306 is suffering from an illness. Defined illness thresholds 348 may be automatically set by machine learning component 324 based on historical illness profile 328 and crowdsourced illness data 342 or may be manually set by user 306. Defined illness thresholds 348 may include two or more threshold levels. For example, defined illness thresholds 348 may include a first threshold indicating that user 306 is entering the illness lifecycle and a second threshold indicating that user 306 has reached a treatment escalation point requiring further medical attention.

Illness manager 322 generates recommendation 350 based on historical illness profile 328 corresponding to user 306, median illness duration and severity 346 of crowdsourced illness data 342, and critical entries 320 in electronic calendar 318 that fall within the mean duration of the illness. Recommendation 350 is a recommended course of action or treatment for user 306, such as, for example, perform critical task “X” at home, reschedule critical event “Y”, take bed rest for two days, increase fluid intake, seek medical professional help, or the like.

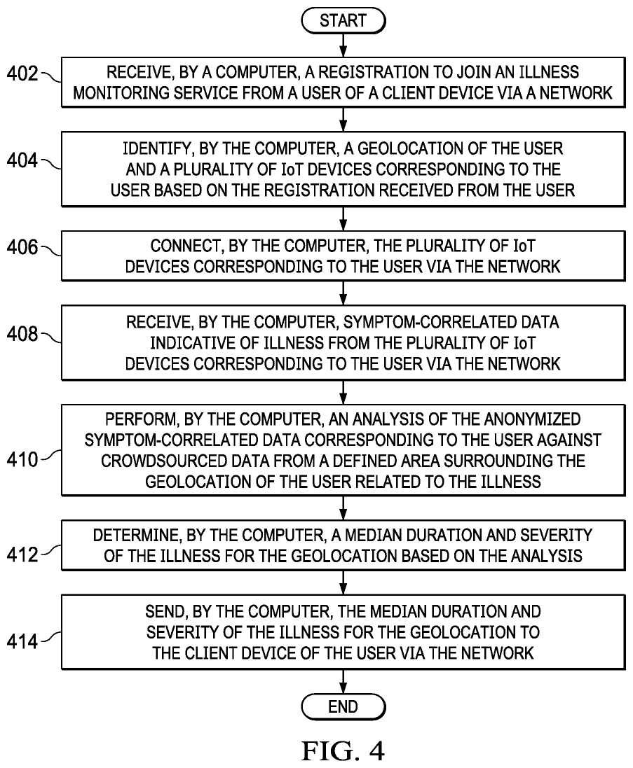

With reference now to FIG. 4, a flowchart illustrating a process for analyzing symptom-correlated data corresponding to a user is shown in accordance with an illustrative embodiment. The process shown in FIG. 4 may be implemented in a computer, such as, for example, server 104 in FIG. 1, data processing system 200 in FIG. 2, or illness monitoring server 302 in FIG. 3.

The process begins when the computer receives a registration to join an illness monitoring service from a user of a client device via a network (step 402). The computer identifies a geolocation of the user and a plurality of IoT devices corresponding to the user based on the registration received from the user (step 404). In addition, the computer connects to the plurality of IoT devices corresponding to the user via the network (step 406).

Further, the computer receives symptom-correlated data indicative of illness from the plurality of IoT devices corresponding to the user via the network (step 408). The computer performs an analysis of the symptom-correlated data corresponding to the user against crowdsourced data from a defined area surrounding the geolocation of the user related to the illness (step 410). The computer determines a median duration and severity of the illness for the geolocation based on the analysis (step 412). The computer sends the median duration and severity of the illness for the geolocation to the client device of the user via the network (step 414). Thereafter, the process terminates.

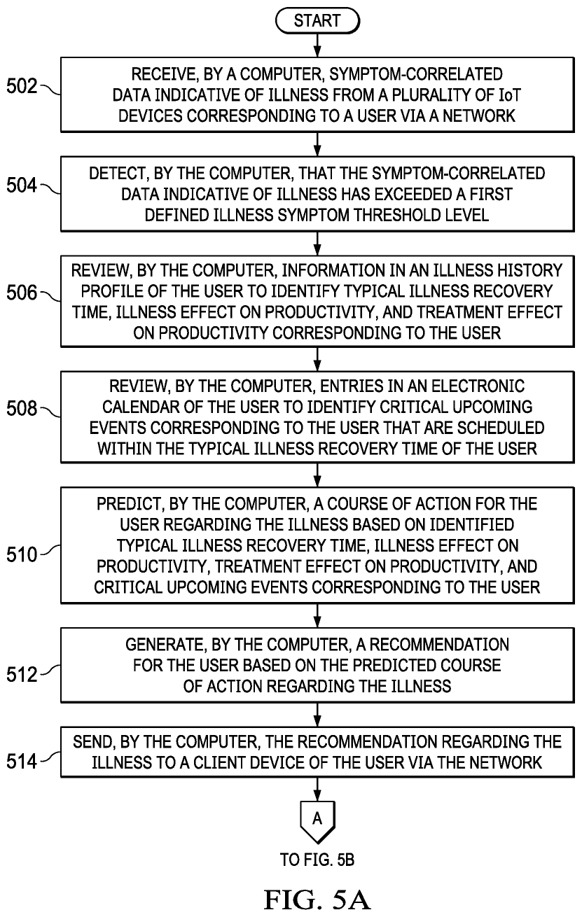

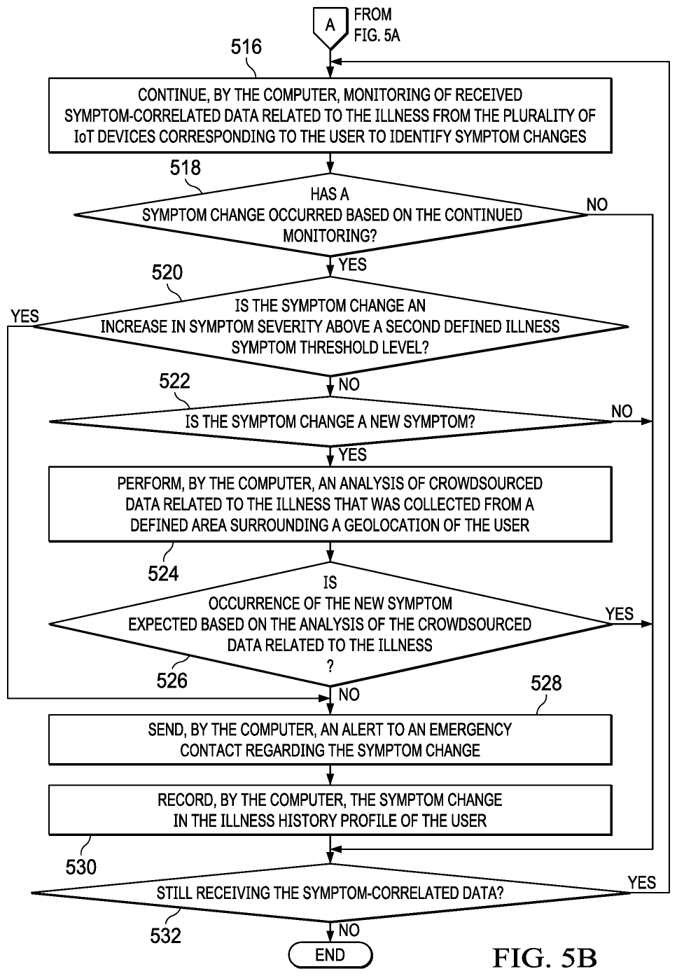

With reference now to FIGS. 5A-5B, a flowchart illustrating a process for predicting a course of action for a user regarding an illness is shown in accordance with an illustrative embodiment. The process shown in FIGS. 5A-5B may be implemented in a computer, such as, for example, server 104 in FIG. 1, data processing system 200 in FIG. 2, or illness monitoring server 302 in FIG. 3.

The process begins when the computer receives symptom-correlated data indicative of illness from a plurality of IoT devices corresponding to a user via a network (step 502). The computer detects that the symptom-correlated data indicative of illness has exceeded a first defined illness symptom threshold level (step 504).

In response to the computer detecting that the symptom-correlated data exceeded the first defined illness symptom threshold level in step 504, the computer reviews information in an illness history profile of the user to identify typical illness recovery time, illness effect on productivity, and treatment effect on productivity corresponding to the user (step 506). In addition, the computer reviews entries in an electronic calendar of the user to identify critical upcoming events corresponding to the user that are scheduled within the typical illness recovery time of the user (step 508).

Afterward, the computer predicts a course of action for the user regarding the illness based on identified typical illness recovery time, illness effect on productivity, treatment effect on productivity, and critical upcoming events corresponding to the user (step 510). The computer generates a recommendation for the user based on the predicted course of action regarding the illness (step 512). The computer sends the recommendation regarding the illness to a client device of the user via the network (step 514).

The computer continues monitoring of received symptom-correlated data related to the illness from the plurality of IoT devices corresponding to the user to identify symptom changes (step 516). The computer makes a determination as to whether a symptom change has occurred based on the continued monitoring (step 518). If the computer determines that no symptom change has occurred based on the continued monitoring, no output of step 518, then the process proceeds to step 532. If the computer determines that a symptom change has occurred based on the continued monitoring, yes output of step 518, then the computer makes a determination as to whether the symptom change is an increase in symptom severity above a second defined illness symptom threshold level (step 520).

If the computer determines that the symptom change is an increase in symptom severity above the second defined illness symptom threshold level, yes output of step 520, then the process proceeds to step 528. If the computer determines that the symptom change is not an increase in symptom severity above the second defined illness symptom threshold level, no output of step 520, then the computer makes a determination as to whether the symptom change is a new symptom (step 522).

If the computer determines that the symptom change is not a new symptom, no output of step 522, then the process proceeds to step 532. If the computer determines that the symptom change is a new symptom, yes output of step 522, then the computer performs an analysis of crowdsourced data related to the illness that was collected from a defined area surrounding a geolocation of the user (step 524). Further, the computer makes a determination as to whether occurrence of the new symptom is expected based on the analysis of the crowdsourced data related to the illness (step 526).

If the computer determines that the occurrence of the new symptom is expected based on the analysis of the crowdsourced data related to the illness, yes output of step 526, then the process proceeds to step 532. If the computer determines that the occurrence of the new symptom is not expected based on the analysis of the crowdsourced data related to the illness, no output of step 526, then the computer sends an alert to an emergency contact regarding the symptom change (step 528). The computer also records the symptom change in the illness history profile of the user (step 530).

The computer makes a determination as to whether the computer is still receiving the symptom-correlated data from the plurality of IoT devices corresponding to the user (step 532). If the computer determines that the symptom-correlated data is still being received from the plurality of IoT devices corresponding to the user, yes output of step 532, then the process returns to step 516 where the computer continues the monitoring of the received symptom-correlated data related to the illness. If the computer determines that further symptom-correlated data is not being received from the plurality of IoT devices corresponding to the user, no output of step 532, then the process terminates thereafter.

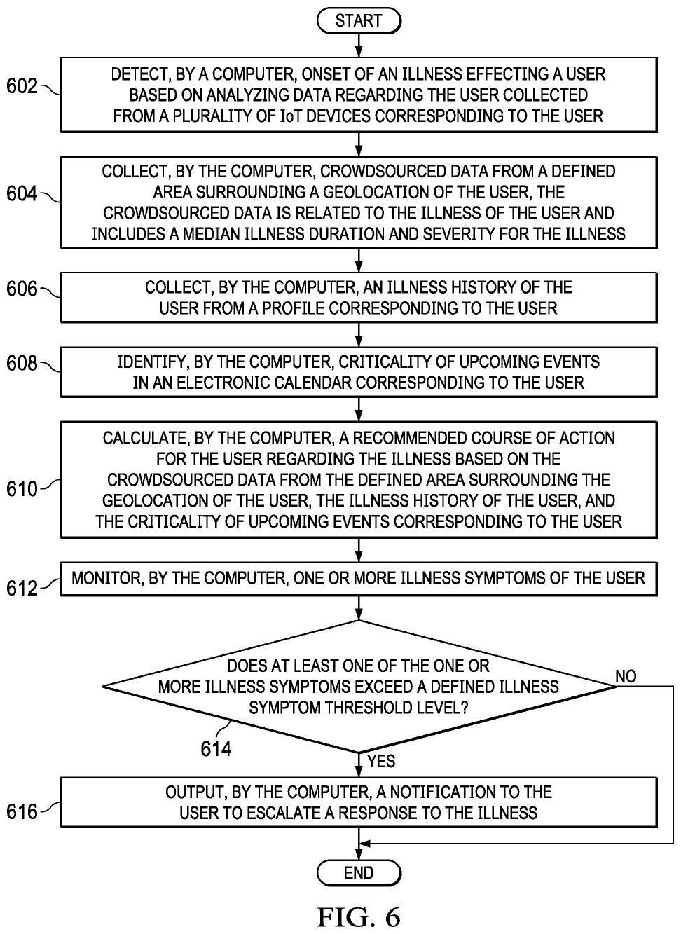

With reference now to FIG. 6, a flowchart illustrating a process for detecting illness onset of a user based on IoT data is shown in accordance with an illustrative embodiment. The process shown in FIG. 6 may be implemented in a computer, such as, for example, server 104 in FIG. 1, data processing system 200 in FIG. 2, or illness monitoring server 302 in FIG. 3.

The process begins when the computer detects onset of an illness effecting a user based on analyzing data regarding the user collected from a plurality of IoT devices corresponding to the user (step 602). In addition, the computer collects crowdsourced data from a defined area surrounding a geolocation of the user (step 604). The crowdsourced data is related to the illness of the user and includes a median illness duration and severity for the illness. Further, the computer collects an illness history of the user from a profile corresponding to the user (step 606).

Furthermore, the computer identifies criticality of upcoming events in an electronic calendar corresponding to the user (step 608). Moreover, the computer calculates a recommended course of action for the user regarding the illness based on the crowdsourced data from the defined area surrounding the geolocation of the user, the illness history of the user, and the criticality of upcoming events corresponding to the user (step 610).

The computer monitors one or more illness symptoms of the user (step 612). The computer makes a determination as to whether at least one of the one or more illness symptoms exceeds a defined illness symptom threshold level (step 614). If the computer determines that none of the one or more illness symptoms exceeds the defined illness symptom threshold level, no output of step 614, then the process terminates thereafter. If the computer determines that at least one of the one or more illness symptoms exceeds the defined illness symptom threshold level, yes output of step 614, then the computer outputs a notification to the user to escalate a response to the illness (step 616). Thereafter, the process terminates.

Thus, illustrative embodiments of the present invention provide a computer-implemented method, computer system, and computer program product for detecting illness onset of a user based on IoT data. The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
detecting onset of an illness effecting a user based on analyzing data regarding the user collected from a plurality of Internet of Things (IoT) devices corresponding to the user;
collecting crowdsourced data from a defined area surrounding a geolocation of the user, the crowdsourced data is related to the illness of the user and includes a median illness duration and severity for the illness;
collecting an illness history of the user from a profile corresponding to the user;
identifying criticality of upcoming events in an electronic calendar corresponding to the user; and
calculating a recommended course of action for the user regarding the illness based on the crowdsourced data from the defined area surrounding the geolocation of the user, the illness history of the user, and the criticality of upcoming events corresponding to the user.

2. The method of claim 1, further comprising:
monitoring one or more illness symptoms of the user;
determining whether at least one of the one or more illness symptoms exceeds a defined illness threshold level; and
responsive to determining that at least one of the one or more illness symptoms exceeds the defined illness threshold, outputting a notification to the user to escalate a response to the illness.

3. The method of claim 1, further comprising:
identifying the plurality of IoT devices corresponding to the user based on a registration received from the user;
connecting to the plurality of IoT devices corresponding to the user via a network; and
receiving symptom-correlated data indicative of the illness of the user from the plurality of IoT devices corresponding to the user via the network.

4. The method of claim 1, further comprising:
performing an analysis of symptom-correlated data corresponding to the user against the crowdsourced data from the defined area surrounding the geolocation of the user related to the illness of the user.

5. The method of claim 1, further comprising:
determining a median duration and severity of the illness of the user for the geolocation of the user based on an analysis of symptom-correlated data corresponding to the user against the crowdsourced data from the defined area surrounding the geolocation of the user related to the illness of the user; and
sending the median duration and severity of the illness for the geolocation to a client device of the user via a network.

6. The method of claim 1, further comprising:
detecting that symptom-correlated data corresponding to the user exceeded a first defined illness symptom threshold level;
reviewing information in the illness history of the user to identify typical illness recovery time, illness effect on productivity, and treatment effect on productivity corresponding to the user; and
reviewing entries in the electronic calendar of the user to identify critical upcoming events corresponding to the user that are scheduled within the typical illness recovery time of the user.

7. The method of claim 1, further comprising:
predicting a course of action for the user regarding the illness based on identified typical illness recovery time, illness effect on productivity, treatment effect on productivity, and critical upcoming events corresponding to the user; and
generating a recommendation for the user based on the course of action regarding the illness; and
sending the recommendation regarding the illness to a client device of the user via a network.

8. The method of claim 1, further comprising:
monitoring received symptom-correlated data related to the illness of the user from the plurality of IoT devices corresponding to the user to identify symptom changes;
determining whether a symptom change has occurred based on the monitoring; and
responsive to determining that a symptom change has occurred based on the monitoring, determining whether the symptom change is an increase in symptom severity above a second defined illness symptom threshold level.

9. The method of claim 8, further comprising:
responsive to determining that the symptom change is an increase in symptom severity above the second defined illness symptom threshold level, sending an alert to an emergency contact regarding the symptom change.

10. The method of claim 8, further comprising:
responsive to determining that the symptom change is not an increase in symptom severity above the second defined illness symptom threshold level, determining whether the symptom change is a new symptom;
responsive to determining that the symptom change is a new symptom, performing an analysis of the crowdsourced data related to the illness of the user that was collected from the defined area surrounding the geolocation of the user; and
determining whether occurrence of the new symptom is expected based on the analysis of the crowdsourced data related to the illness of the user.

11. The method of claim 10, further comprising:
responsive to determining that the occurrence of the new symptom is not expected based on the analysis of the crowdsourced data related to the illness of the user, sending an alert to an emergency contact regarding the symptom change and recording the symptom change in the illness history of the user.

12. A computer system comprising:
a bus system;
a storage device connected to the bus system, wherein the storage device stores program instructions; and
a processor connected to the bus system, wherein the processor executes the program instructions to:
detect onset of an illness effecting a user based on analyzing data regarding the user collected from a plurality of Internet of Things (IoT) devices corresponding to the user;
collect crowdsourced data from a defined area surrounding a geolocation of the user, the crowdsourced data is related to the illness of the user and includes a median illness duration and severity for the illness;
collect an illness history of the user from a profile corresponding to the user;
identify criticality of upcoming events in an electronic calendar corresponding to the user; and
calculate a recommended course of action for the user regarding the illness based on the crowdsourced data from the defined area surrounding the geolocation of the user, the illness history of the user, and the criticality of upcoming events corresponding to the user.

13. The computer system of claim 12, wherein the processor further executes the program instructions to:
monitor one or more illness symptoms of the user;
determine whether at least one of the one or more illness symptoms exceeds a defined illness threshold level; and
output a notification to the user to escalate a response to the illness in response to determining that at least one of the one or more illness symptoms exceeds the defined illness threshold.

14. The computer system of claim 12, wherein the processor further executes the program instructions to:
identify the plurality of IoT devices corresponding to the user based on a registration received from the user;
connect to the plurality of IoT devices corresponding to the user via a network; and
receive symptom-correlated data indicative of the illness of the user from the plurality of IoT devices corresponding to the user via the network.

15. The computer system of claim 12, wherein the processor further executes the program instructions to:
perform an analysis of symptom-correlated data corresponding to the user against the crowdsourced data from the defined area surrounding the geolocation of the user related to the illness of the user.

16. A computer program product comprising a computer readable storage medium having program instructions embodied therewith, the program instructions executable by a computer to cause the computer to perform a method comprising:
detecting onset of an illness effecting a user based on analyzing data regarding the user collected from a plurality of Internet of Things (IoT) devices corresponding to the user;
collecting crowdsourced data from a defined area surrounding a geolocation of the user, the crowdsourced data is related to the illness of the user and includes a median illness duration and severity for the illness;
collecting an illness history of the user from a profile corresponding to the user;
identifying criticality of upcoming events in an electronic calendar corresponding to the user; and
calculating a recommended course of action for the user regarding the illness based on the crowdsourced data from the defined area surrounding the geolocation of the user, the illness history of the user, and the criticality of upcoming events corresponding to the user.

17. The computer program product of claim 16 further comprising:
monitoring one or more illness symptoms of the user;
determining whether at least one of the one or more illness symptoms exceeds a defined illness threshold level; and
responsive to determining that at least one of the one or more illness symptoms exceeds the defined illness threshold, outputting a notification to the user to escalate a response to the illness.

18. The computer program product of claim 16 further comprising:
identifying the plurality of IoT devices corresponding to the user based on a registration received from the user;
connecting to the plurality of IoT devices corresponding to the user via a network; and
receiving symptom-correlated data indicative of the illness of the user from the plurality of IoT devices corresponding to the user via the network.

19. The computer program product of claim 16 further comprising:
performing an analysis of symptom-correlated data corresponding to the user against the crowdsourced data from the defined area surrounding the geolocation of the user related to the illness of the user.

20. The computer program product of claim 16 further comprising:
determining a median duration and severity of the illness of the user for the geolocation of the user based on an analysis of symptom-correlated data corresponding to the user against the crowdsourced data from the defined area surrounding the geolocation of the user related to the illness of the user; and
sending the median duration and severity of the illness for the geolocation to a client device of the user via a network.
